(19) United States
(12) Patent Application Publication
(10) Pub. No.: US 2024/0415665 A1
(43) Pub. Date: Dec. 19, 2024

(54) Humeral Implant with Cannulation and Method

(71) Applicant: Steven C. Chudik, Western Springs, IL (US)

(72) Inventor: Steven C. Chudik, Western Springs, IL (US)

(21) Appl. No.: 18/211,396

(22) Filed: Jun. 19, 2023

(51) Int. Cl. A61F 2/40; A61F 2/30; A61F 2/46

(52) U.S. Cl. CPC A61F 2/4003; A61F 2/30749; A61F 2/4014; A61F 2/4081; A61F 2/4612; A61F 2002/4007; A61F 2002/4022; A61F 2002/4085; A61F 2220/0016; A61F 2220/0033; A61F 2220/0041

(57) ABSTRACT

A humeral implant is disclosed. The implant has a bone fixation component and an articular surface component. The bone fixation component is configured for fixation to an articular portion of a proximal humerus and has a cannulation extending through the bone fixation component. The articular surface component is connectable to the bone fixation component and has an articular surface. A method of shoulder replacement is also disclosed.

### FIELD OF THE INVENTION

The present invention relates in general to humeral implants.

### BACKGROUND OF THE INVENTION

Orthopaedic surgeons often perform joint replacement surgery on patients who suffer pain and physical limitations caused by joint surfaces which have degenerative, traumatic, or other pathologic damage. The success of replacement surgery is related to the degree of morbidity associated with the surgical technique and also to the ability of the surgery to restore the natural anatomy and biomechanics of the joint. The present inventor recognized the need for improved rates of surgical success through an improved implant and method.

### BRIEF SUMMARY OF THE INVENTION

A new humeral implant is disclosed. In some embodiments, the implant has a bone fixation component and an articular surface component. The bone fixation component is configured for fixation to an articular portion of a proximal humerus. The bone fixation component has a cannulation extending through the bone fixation component. The articular surface component is connectable to the bone fixation component and has an articular surface. In some embodiments, the cannulation traverses both components of the humeral implant. In some embodiments, the implant has a stem that has a stem cannulation.

The cannulation(s) may be configured to allow transhumeral operation and/or passage of instruments, screws, or other implant components to access the glenohumeral joint of a shoulder and prepare the glenoid bone, or place, repair, or replace a glenoid implant.

In some embodiments, the bone fixation component and the articular surface component are a unitary articular humeral component where a cannulation extends through the articular humeral component from a bone fixation side through an articular surface of the articular humeral component.

A method of shoulder replacement is disclosed. In some embodiments, a humeral implant component is placed at a prepared portion of a proximal humerus. An instrument is operated transhumerally through the cannulation. In some embodiments, the component is a stem with a cannulation, a bone fixation component with a cannulation, an articular surface component with or without cannulation, and/or a spacer.

In some embodiments the instrument is operated to prepare the glenoid to receive a glenoid implant. In some embodiments, the instrument is operated to remove a prior glenoid implant. In some embodiments, the instrument is operated to fix a glenoid implant to a prepared glenoid. In some embodiments, the instrument is operated to place a component of the glenoid implant in the glenoid.

Other features and advantages of this invention will be apparent to orthopaedic surgeons and other persons who are skilled in the art of shoulder repair and reconstruction, particularly after reviewing the following detailed description of the invention and the embodiments thereof, from the claims, and from the accompanying drawings.

### BRIEF DESCRIPTION OF THE DRAWINGS

FIG. 1A is a perspective view of a portion of a humerus bone with a common arthritic deformity of the humeral head.

FIG. 1B is a perspective view of a portion of a scapula bone with a glenoid process.

FIG. 2A is a side section view of a traditional humeral implant positioned in a prepared humerus bone.

FIG. 2B is a side, partially transparent, view of another common humeral implant utilizing fins in a metaphyseal position of a prepared humerus bone.

FIG. 3 is a side perspective view of a first embodiment humeral implant of the invention with a concave articular surface component for reverse shoulder arthroplasty.

FIG. 4A is a side perspective view a second embodiment humeral implant of the invention with a convex articular surface component for anatomic shoulder arthroplasty.

FIG. 4B is a top view of the articular humeral component of the humeral implant of FIG. 4A showing the implant's elliptical shape which incorporates different axial and longitudinal radii of curvature.

FIG. 5A is an exploded side perspective view of the assembly of the humeral implant of FIG. 3.

FIG. 5B is an exploded side perspective view of the assembly of the humeral implant of FIG. 4A.

FIG. 5C is a side perspective view of the shell of the humeral implant of FIGS. 3 and 4A with a peripheral rim and fin(s) to resist subsidence and rotation respectively.

FIG. 5D is a top view of an embodiment of the shell of the humeral implant of FIG. 3 with a cannulation for accessing a glenoid and glenohumeral joint.

FIG. 5E is a top view of an embodiment of an articular surface component of the humeral implant of FIG. 3 with an optional cannulation for accessing a glenoid and glenohumeral joint;

FIG. 5F is a side perspective view of the articular surface component of the humeral implant of FIG. 3 with an optional cannulation for accessing a glenoid and glenohumeral joint.

FIG. 5G is a bottom view of an embodiment of an articular surface component of the humeral implant of FIG. 4A.

FIG. 5H is a top view of the articular surface component of FIG. 5G.

FIG. 5I is a side perspective view of the humeral implant of the invention with the addition of an intervening spacer.

FIG. 5J is a side perspective view of the spacer for the humeral implant of FIG. 5I.

FIG. 5K is a top view of the spacer of FIG. 5J for the humeral implant of FIG. 5I.

FIG. 5L is a side perspective view of the cannulated stem of FIGS. 3 and 4A.

FIG. 6A is a perspective view of the shell of the humeral implant of FIGS. 3 and 4A positioned in a prepared humerus bone with an associated lateral buttress washer-plate secured to the bone with screws engaging the implant with a stem.

FIG. 6B is a second perspective view of the ingrowth shell of FIG. 6A.

FIG. 7A is a side perspective view of a handle arranged to hold the head of a humeral reamer.

FIG. 7B is a side view of the humeral reamer and handle of FIG. 7A for preparing a humerus for the installation of the humeral implant of FIG. 3 or 4A.

FIG. 7C is a bottom view of the humeral reamer of FIG. 7B.

FIG. 7D is a side view of a portion of the reamer shaft with engagement tip of the shaft and centering handle sleeve to interact with the head of the humeral reamer of FIGS. 7B, 7C, and 7E.

FIG. 7E is a perspective view of the humeral reamer of FIG. 7B and shaft of FIG. 7D arranged through a transhumeral passage in the humerus for a step to prepare a humerus for the installation of the new convertible humeral implant.

FIG. 7F is a perspective view of the prepared humerus and transhumeral sheath after steps to prepare the humerus for the installation of the humeral implant of FIG. 3 or 4A where the reamer shaft and head of FIG. 7E are disengaged and removed.

FIG. 8A is an elevational view of a modular glenoid reamer and handle.

FIG. 8B is a second elevational view of the modular glenoid reamer of FIG. 8A.

FIG. 8C is a side perspective view of a handle arranged to hold the head of the glenoid reamer of FIG. 8A.

FIG. 9A is a perspective view of the glenoid reamer of FIG. 8A and shaft of FIG. 7D arranged through a transhumeral passage and the cannulation in the shell of the humeral implant of FIG. 3, 4A, or 5I in a humerus to prepare the glenoid for a glenoid implant.

FIG. 9B is a perspective view of the glenoid reamer of FIG. 8A and shaft of FIG. 7D arranged through a transhumeral passage and the cannulation in the shell and, optionally, in the articular surface component in a humerus to prepare the glenoid for a glenoid implant.

FIG. 9C is a second perspective view of the glenoid reamer and shaft of FIG. 7D arranged through a transhumeral passage, the shell I in the humerus to prepare the glenoid for a glenoid implant.

FIG. 9D is a third perspective view of the humerus and glenoid of 9A after the glenoid and humerus have been prepared by humeral and glenoid reamers of FIGS. 7A, 7B, 7C, 7D, 7F and 8A, 8B, 8C through a transhumeral passage, the shell.

FIGS. 9E, 9F, and 9G are third perspective views of the humerus and glenoid of 9A where a component of the glenoid implant is inserted and removed through a transhumeral passage, the shell and, optionally, the articular surface component, spacer and stem of the humeral implant of FIG. 3,4A, or 5I in the humerus.

### DETAILED DESCRIPTION OF THE INVENTION

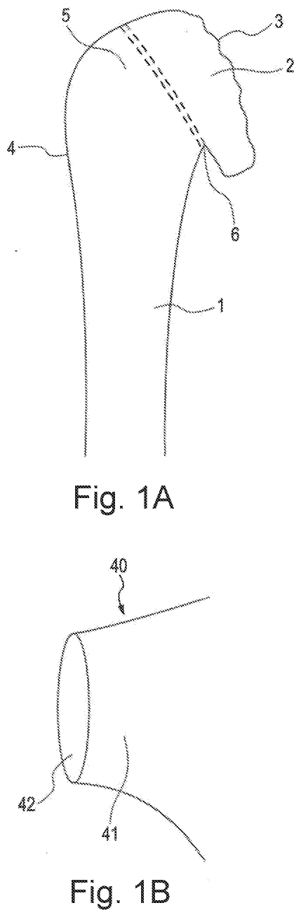

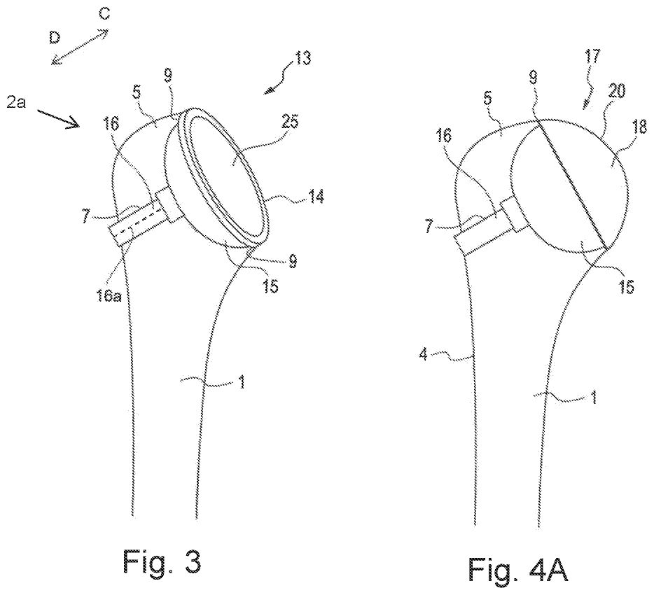

Shoulder arthroplasty surgery traditionally requires transection of the subscapularis tendon, dislocation of the shoulder joint and resection of the head 2 of the humerus bone 1 at the level of plane 6 of the anatomic neck (FIG. 1A) to gain sufficient access to prepare and resurface the glenoid 40 (FIG. 1B). Using a novel transhumeral method allows for proper access to the glenoid while sparing the subscapularis tendon and avoiding dislocation. Novel humeral implants 13, 17 (FIGS. 3; 4A, B; 5A, B, C, D, E; 6A, B) are usable with this novel transhumeral method, which provides a minimally invasive technique, but can also be used with traditional surgical techniques.

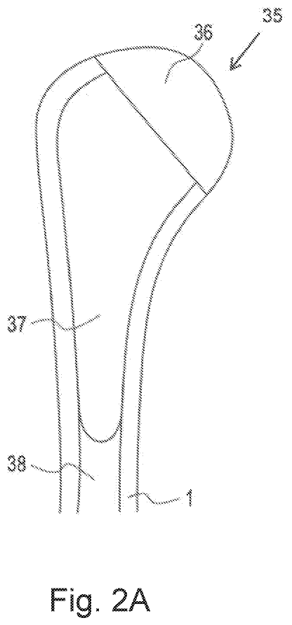

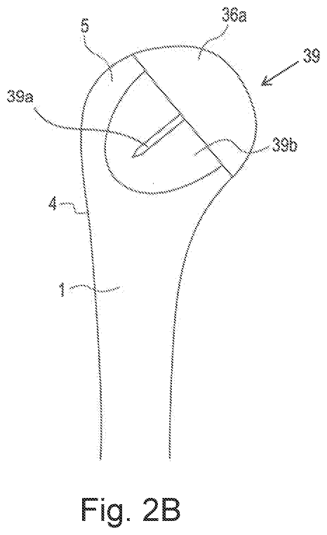

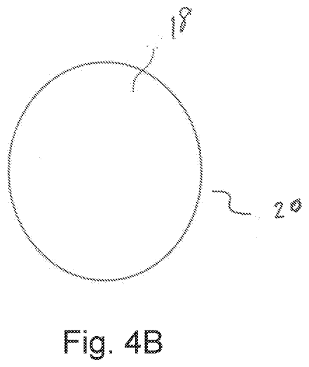

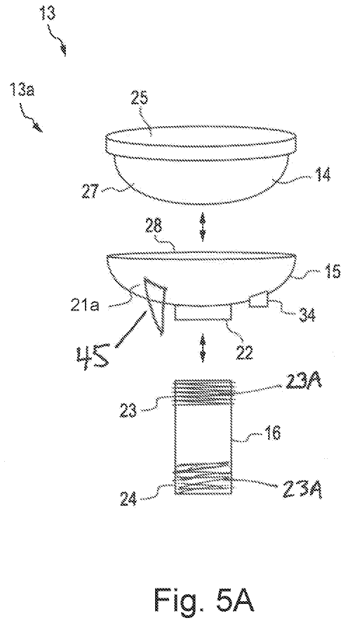

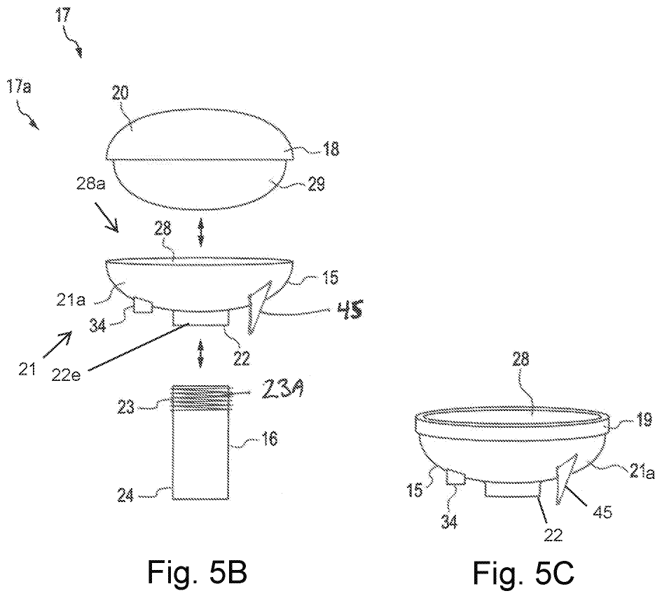

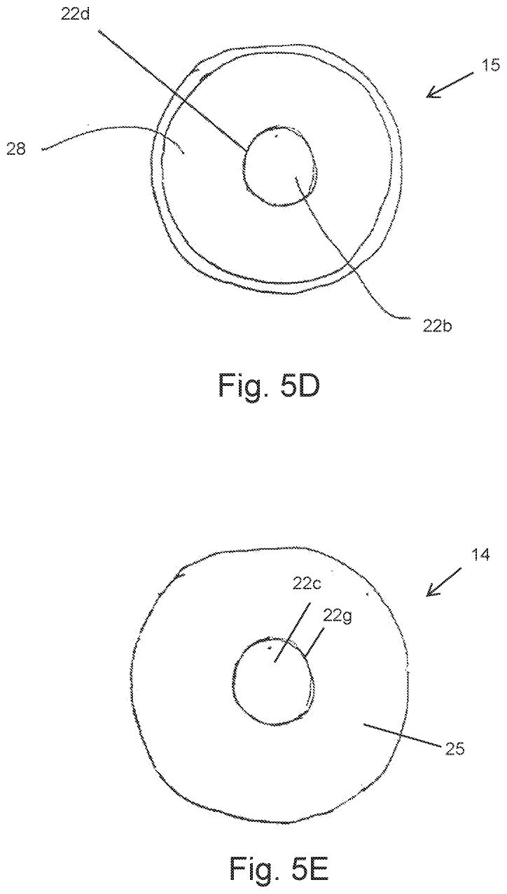

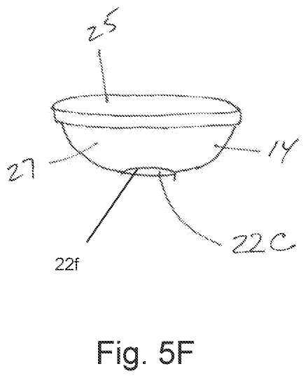

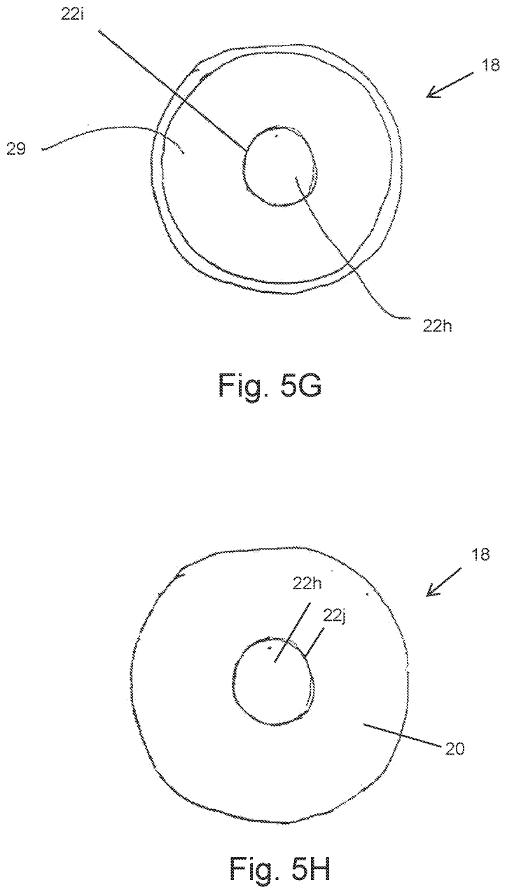

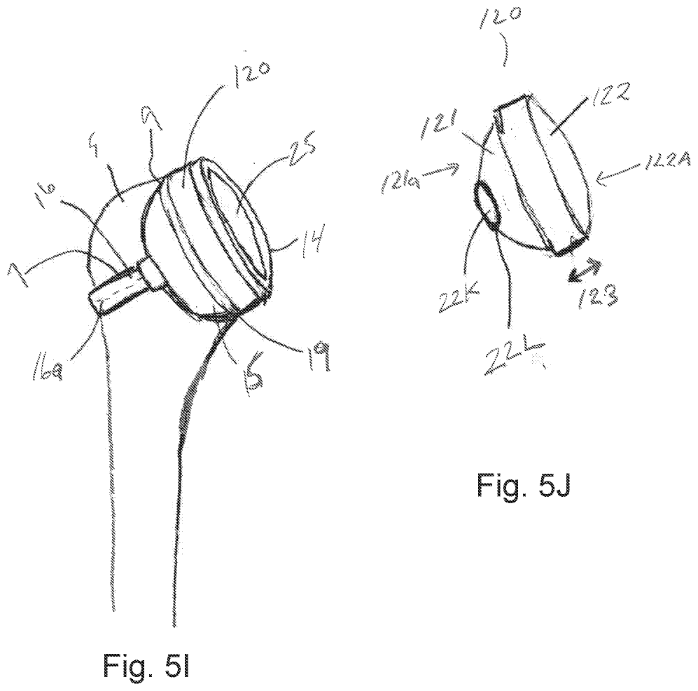

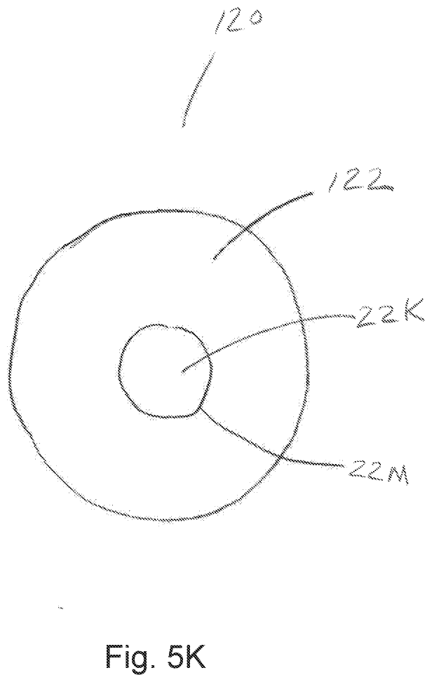

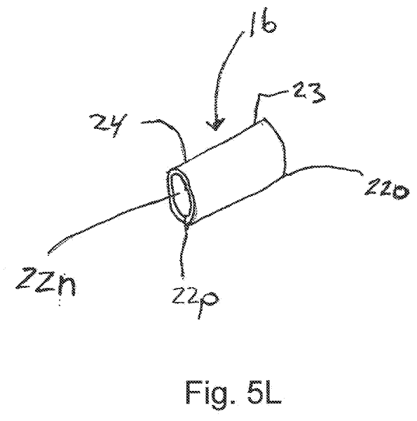

In the novel transhumeral method, the components of the new humeral implants 13, 17, as opposed to other implants 35, 39 (FIGS. 2A, 2B,), can be more easily inserted without cutting the rotator cuff nor dislocating the shoulder joint through non-bony soft-tissue passageways with or without the assistance of transhumeral tunnel 7 to the shoulder. The novel humeral implants 13, 17 have at least cannulation 20a, 22b in the shell 15, and optionally a cannulation 22c in the articular surface component 14, 18, and optionally a cannulation 22n in the stem 16 that allows transhumeral access to the glenoid 40 and glenohumeral joint (FIGS. 5D, 5E, 5F). The novel humeral implants 13,17 avoid the limitations associated with traditional humeral implants 35 having round, not oval shaped, articular components 36 with offset intramedullary canal stems 37 designed to be fit in the intramedullary canal 38 of the humerus 1 (FIG. 2A). Limitations associated with traditional humeral implants 35 include but are not limited to complicated implant removal and difficulty in obtaining anatomic parameters of humeral version, height, depth, inclination, and radius of curvature. In some embodiments, the articulating surface 20 comprises different anterior-to-posterior and superior-to-inferior radii of curvature more the native humeral head (FIG. 4A, B). New humeral implants 13, 17 (FIGS. 3, 4, 5A, 5B) also allow convertibility not afforded by prior art implants, such as implant 39.

In some embodiments, the novel humeral implants 13, 17 comprise an optional stem 16 that provides slidable engagement with the bone of the humeral metaphysis 5 and/or non-articular lateral bony cortex 4 of the humerus 1 which allows for more physiologic loading of the humeral bone 1, 2, 3, 5 than with other humeral implants 35, 39 and their fixed stems 37, 39a. Additionally, in the case of a periprosthetic humeral fracture complication, the design of the new humeral implants 13, 17 will provide for a pattern of proximal humeral fracture at the end of the unfixed stem 16, more easily managed than those predicted to occur with humeral implants 35, 39 and which does not compromise implant fixation.

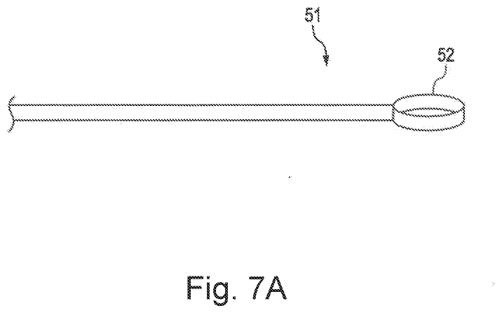

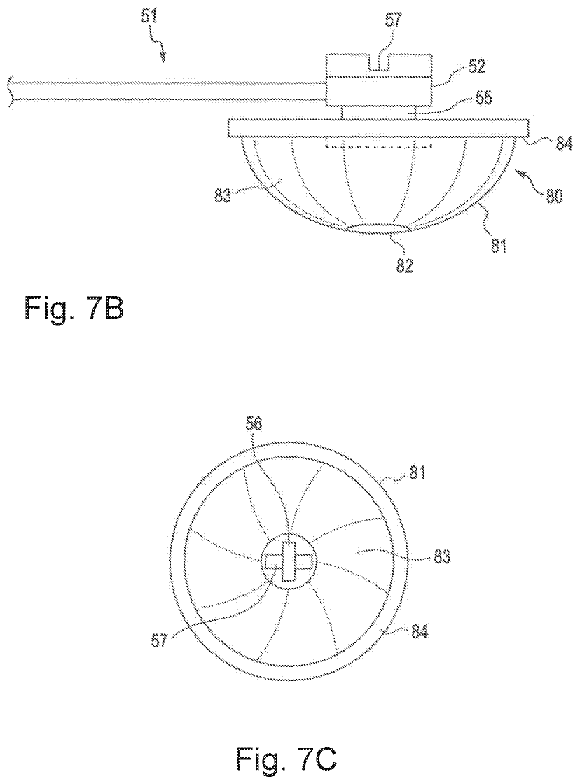

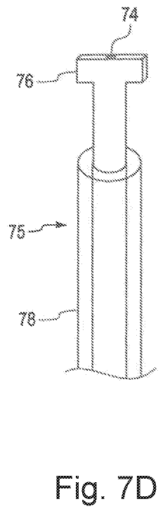

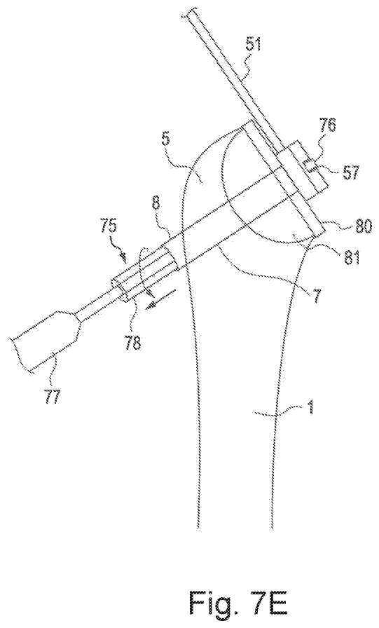

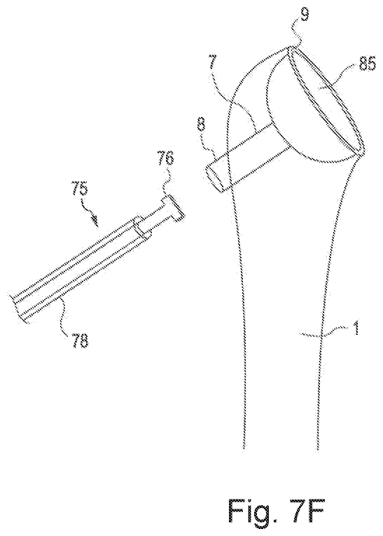

The worn articular surface 3 of the humerus 1 can be prepared with a modular transhumeral reamer 80 (FIGS. 7A, B, C, D, E, F)). The modular transhumeral reamer 80 has a transhumeral reamer shaft 75 and modular cutting reamer head 81 with an attached handle 51 that still allows the reamer head 81 to spin around its central axle 55 in the ring end 52 while it is held in position by the handle 51. The reamer 80, the reamer head 81 which reams into the vault of the humeral metaphysis to allow for new convertible humeral implants 13, 17 (FIGS. 7A, B, C, D, E, F).

Each convertible humeral implant 13, 17 comprises an articular humeral component 13a, 17a and an optional stem 16. The articular humeral component 17a comprises a bone fixation component or shell 15 and an articular surface component 18. The articular surface component 18 comprises a convex articulating surface 20 and a coupling surface 29. The articular humeral component 13a comprises the shell 15 and an articular surface component 14. The articular surface component 14 comprises a concave articulating surface 25 and a coupling surface 27. The optional stem 16 does not reside in nor rely on the intramedullary canal 38 for fixation (FIGS. 3; 4A, B; 5A, B, C, D, E). In some embodiment, the implants 13, 17 are modular at least in that the stem is connectable to the articular humeral component 13a, 17a. The implants 13, 17 obtains stability from contact with good quality bone from the metaphyseal bone 5 and non-articular lateral bony cortex 4 of the humerus 1. The stem 16 is positioned in a transhumeral tunnel 7 along a central axis of the humeral metaphysis 5.

Implants 13, 17 allow interchangeability of articular surface component 14, 18 with either the convex or concave articulating surface 20, 25 to allow easy conversion between anatomic shoulder arthroplasty and reverse shoulder arthroplasty (FIGS. 3 and 4A, B). The novel convertible humeral implants 13, 17 each comprise a shell 15 and optionally the stem 16 (5A, B, C, D, E). The articular surface components 14, 18 comprise coupling surfaces 27, 29 that reversibly connect to the coupling surface 28 of the shell 15 (FIGS. 3, 4A, 4B, 5A, 5B, 5C, 5D, 5E). The coupling surfaces 27, 29 of the articular surface component 14, 18 may possess variable geometry and one of a variety of reversible coupling mechanisms, such as threads or press fit. The articular surface component 14, 18 may be modular and possess structural voids to reduce weight. The shell 15 may comprise variable protruding geometry with a bony ingrowth surface 21a, a peripheral rim 19 to rest upon the prepared cortical rim 9 of the proximal humerus 2a and fins or other prominences 45 extending from the protruding side of the shell 15 (FIGS. 3, 4A, 4B, 5A, 5B, 5C, 5D, 5E, 6A, 6B, 7F).

The shell 15 comprises at first cannulation 22b, such as shown in FIG. 5D. The coupling surface 28 of the coupling side 28a of the shell has a first aperture 22d in communication with the cannulation 22b and the fixation side 21 has a fixation surface(s) 21a with a second aperture 22e in communication with the cannulation 22b. The cannulation 22b extends through the shell 15 from the fixation side 21 to the coupling side 28a.

In some embodiments, the cannulation is defined by one or more interior sidewalls that extend from the coupling surface 28 to the fixation surface 21a. In some embodiments, the one or more interior walls are cylindrical throughout the cannulation and circular in cross-section. In some embodiments, when a stem 16 is used, the cannulation 22b is co-axial with the stem. The first cannulation may be at a radial center of the shell 15, and in particular at a bottom and radial center of the coupling surface 28.

In some embodiments, the articular surface component 14, 18 comprises a second cannulation 22c, 22h. The second cannulation 22c, 22h is aligned with the first cannulation 22b when the articular surface component 14, 18 is connected to the bone fixation component 15. In some embodiments, the articular surface component 14, 18 does not have a second cannulation 22c, 22h.

The coupling surface 27 comprises an aperture 22f in communication with the second cannulation 22c and the articulating surface 25 comprises an aperture 22g in communication with the second cannulation 22c. The second cannulation 22c extends through the articular surface component from the articulating surface 25 to the coupling surface 27. The second cannulation 22c may be at a radial center of the articular surface component 14, 18, and in particular at a bottom and radial center of the articulating surface 25.

In some embodiments, the second cannulation 22c is defined by one or more interior sidewalls that extend from the coupling surface 27 to the articulating surface 25. In some embodiments, the one or more interior walls are cylindrical throughout the second cannulation and circular in cross-section. The second cannulation 22c is aligned and co-axial with the first cannulation 22b when the articular surface component is seated in the bone fixation component 15. In some embodiments, when a stem 16 is used, the second cannulation 22c is co-axial with the stem.

The coupling surface 29 comprises an aperture 22i in communication with the second cannulation 22h of articular surface component 18 and the articulating surface 20 comprises an aperture 22j in communication with the second cannulation 22h, as shown in FIGS. 5G and 5H. The second cannulation 22h extends through the articular surface component 18 from the articulating surface 20 to the coupling surface 29. The second cannulation 22h may be at a radial center of the articular surface component 18.

In some embodiments, the second cannulation 22h is defined by one or more interior sidewalls that extend from the coupling surface 29 to the articulating surface 20. In some embodiments, the one or more interior walls are cylindrical throughout the second cannulation and circular in cross-section. The second cannulation 22h is aligned and co-axial with the first cannulation 22b when the articular surface component 18 is seated in the bone fixation component 15. In some embodiments, when a stem 16 is used, the second cannulation 22h is co-axial with the stem.

In some embodiments, the first cannulation 22b and second cannulation 22c, 22h, and cannulations 22k 22n comprise a width or diameter sized to allow the transhumeral passage through the cannulation(s) of instruments, screws, and other implants or implant components to prepare the glenoid bone or place, repair, or replace a glenoid implant in a shoulder. In some embodiments, the width or diameter is in the range of 1 mm to 25 mm, or 5 mm to 15 mm, or 5 mm to 10 mm, inclusive of the end values. Because of varying anatomy and deformity of the glenohumeral joint from patient to patient, the humeral implants 13,17 can be configured in in different small to large anatomically appropriate sizes which can be customized, such as by using advanced imaging data and 3-dimensional printing. The articular surface components 14, 18 also can be configured in a variety of height, length, width, and curvature options to restore proper variable glenohumeral anatomic relationships.

In some embodiments and applications, when performing a reverse total shoulder arthroplasty or converting from an anatomic to a reverse total shoulder arthroplasty configuration, the humeral implants 13,17 may optionally comprise a spacer 120 to restore proper length and tension of the spanning soft-tissues of the glenohumeral joint (FIGS. 5I, J, K). The spacer 120 has two sides 121a, 122a with respective coupling surfaces 121, 122. The two coupling surfaces 121, 122 couple with the coupling surface 28 of the shell 15 of the humeral implants 13, 17 and the coupling surfaces 27, 29 of the articular surface components 14, 18. Therefore the spacer joints the shell 15 and the articular surface component 14, 18. In some embodiments, the spacer releasably or reversibly couples the shell 15 and the articular surface component 14, 18. The spacer 120 comprises a height 123. The height 123 can be selected to correspond to the desired positioning articular component. Further, multiple spacers can be provided of varying heights 123, allowing the selection of the spacer 120 with the desire height for a given patient.

In some embodiments, the spacer 120 has a cannulation 22k such as shown in FIGS. 5J and 5K. The first coupling surface 121 of the first coupling side 121a of the spacer 120 has a first aperture 22L in communication with the cannulation 22k and the second coupling side has a second coupling surface 122 with a second aperture 22m in communication with the cannulation 22k. The cannulation 22k extends through the spacer 120 from the first coupling side 121a to the second coupling side 122a.

In some embodiments, the cannulation 22k is defined by one or more interior sidewalls that extend from the first coupling surface 121 to the second coupling surface 122. In some embodiments, the one or more interior walls are cylindrical throughout the cannulation and circular in cross-section. In some embodiments, when a stem 16 is used, the cannulation 22k is co-axial with the stem. The cannulation 22k may be at a radial center of the spacer 120, and in particular at a bottom and radial center of the second coupling surface 122.

In some embodiments, each humeral implant 13, 17 may be formed such that articular humeral component 13a, 17a is a unitary component where the articular surface component 14, 18, is formed together with the shell 15 as a unitary component, with or without a spacer 120 formed as a part of the unitary component. When humeral component 13a, 17a is a unitary component the portion thereof corresponding to the articular surface component 14, 18 will have a cannulation, such as second cannulation 22c, 22h, aligned and co-axial with the first cannulation 22b, so that the cannulation extends through the humeral component 13a, 17a from the articulating surface 20, 25 to fixation side 21.

The stem 16 is positioned to reside in the transhumeral tunnel 7 along a central axis of the humeral metaphysis 5 transverse to the plane 6 of the humeral anatomic neck, such as shown in FIGS. 3 and 4A. In some embodiments, the stem 16 and the transhumeral tunnel 7 are perpendicular to the plane 6 of the humeral anatomic neck. The stem 16 can possess a first end 23 with a coupling feature or portion 23a to engage the opposite coupling feature or portion 22 of the humeral implants 13, 17. The cannulation 22b can extend to or through the coupling feature 22. In some embodiments, an interior wall of the cannulation 22b at a terminal end of the cannulation at or adjacent second aperture 22e opposite of the coupling surface 28, comprises a first coupling feature, and an end of the stem comprises a second coupling feature. The stem is connectable at the interior wall between the first and second coupling features. In some embodiments, the coupling features 22, 23a comprise threads, a press fit, or a morse taper, or other suitable coupling mechanism to join the stem to the articular humeral component 13a, 17a. It is possible for the male or female counterpart to be on either the stem 16 or the articular humeral component 13a, 17a, with the opposite counterpart on the other of the stem 16 or the articular humeral component 13a, 17a. In some embodiments, in the case of the coupling feature 22 comprising threads, the coupling feature 22 is a receiver having a peripheral wall surrounding an opening for receiving the stem 16, the peripheral wall comprises interior threads for engaging the threads at the first end 23 of the stem 16. In some embodiments, the stem 16 comprises a second coupling feature 23a, such as threads or a press-fit, at a second end 24 of the stem opposite the first end as shown for stem 16 in FIG. 5A. In some embodiments, the second end of the stem 16 does not comprise a second coupling feature but instead may be smooth, such as shown in FIG. 5B. The second end of the stem 16, whether comprising a second coupling feature or not, obtains contact and stability against the bone of non-articular lateral bony cortex 4 and/or the bone of the humeral metaphysis 5 (FIGS. 3, 4A), when implanted.

In various embodiments, the stem 16 may comprise different lengths. In some embodiments, the stem 16 comprises a length such that the stem engages only the bone of the metaphysis 5. In some embodiments, the stem 16 comprises a length such that the stem 16 extends beyond the metaphysis 5 to also engage the non-articular lateral bony cortex 4, and the stem 16 may or may not extend past the lateral end of the lateral bony cortex 4. Therefore, the stem 16 can be contained within the transhumeral tunnel 7 or may extend out of the transhumeral tunnel 7 at the lateral end of the lateral bony cortex 4. In some embodiments, the stem 16 comprises a length in the range of 1 cm and 6 cm, inclusive. In some embodiments, the stem 16 comprises a length that is configured to engage sufficient structural bone of the humerus to resist non-longitudinal displacement of the stem.

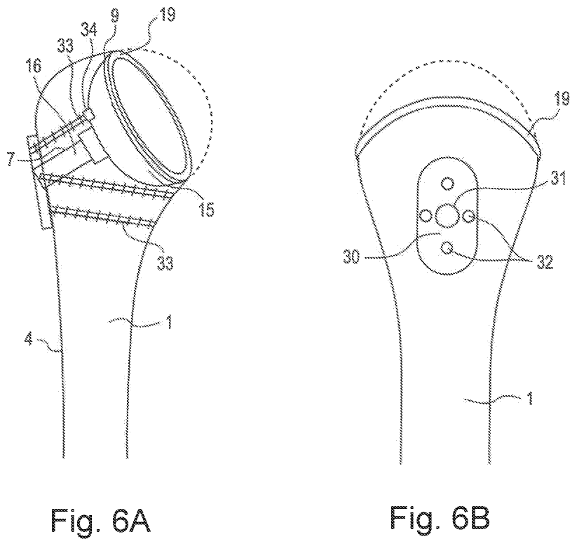

In some embodiments and applications, the stem 16 also engages a coupling site 31 of the washer-plate 30 on the non-articular lateral bony cortex 4 of the humerus to improve implant stability (FIGS. 6A, 6B). In some embodiments, the coupling site 31 comprises threads on a wall of the aperture where the stem 16 is received. Therefore, when the second end 24 of the stem comprise threads, the thread the stem engage the thread of the coupling site 31.

In some embodiment and applications, the stems 16 are configured for slidable engagement with the non-articular lateral bony cortex 4 of the humerus, the washer-plate 30, and/or the bone of the humeral metaphysis 5 to allow more physiologic load transmission of joint compressive forces to the remainder of the bone of the humerus 1 through the humeral implants 13, 17. The stem 16 is configured not to be axially fixed within the transhumeral tunnel 7, but instead to slide within the transhumeral tunnel 7, including after implantation surgery is complete, i.e. post-surgery. The stem 16 can slide longitudinally within the transhumeral tunnel 7 and axially along a longitudinal axis 16a of the stem, in the directions C and D of FIG. 3. The stem 16 can be limited in its sliding range of motion in the first direction D by the articular humeral component 13a, 17a engagement with the humeral head. The stem does not axially shelter the load born by the articular humeral component from the surrounding proximal humeral bone in direction D. This non-load-sheltering aspect of the stem contributes to more physiologic load transmission of joint compressive forces to the remainder of the bone of the humerus 1 through the humeral implants 13, 17. In some embodiments, the width or diameter of the stem 16 less than the width or diameter of the transhumeral tunnel 7 and therefore stem does not friction fit against the wall(s) of the transhumeral tunnel 7 and therefore, the stem is slidable within the transhumeral tunnel 7, including post-surgery. In some embodiments, the stem 16 can be provided with a smooth exterior, such as a smooth exterior surface below the coupling feature 23a at the first end 23 of the stem 16 shown in FIG. 5B. And, at least the second end 24 may be smooth, such as shown in FIG. 5B. In some embodiments, the stem 16 has a uniform width and cross-section along the longitudinal length of the stem or a portion thereof.

In some embodiments, the second end 24 of the stem 16 is enlarged to prevent movement toward the articular humeral component 13a, 17a in the direction C, where the enlarged portion engages with a portion of the tunnel 7, which is narrower than the enlarged portion, at least in part, to prevent further movement in the direction C. In some embodiments, the stem 16 comprises ridges, fins, and/or unidirectional ridges and these features of the stem do not limit movement of the stem away from the articular humeral component along its axis, such as in the direction D. In some embodiments, the stem 16 may be considered nonadherent within the tunnel 7 in that it allows movement away from the articular humeral component, such as in the direction D. The range of movement in a first direction of the stem 16 within the tunnel may be limited by the articular humeral component's 13a, 17a engagement with the prepared portion of a proximal humerus 2a via the connection between the stem and the articular humeral component 13a, 17a. In some embodiments, the range of movement of the stem in a second direction, opposite the first direction, in the tunnel 7 may be limited by an enlarged second end 24 of the stem and its engagement with the tunnel or another fixture.

In some embodiments, the washer-plate 30 does not have a coupling site and instead a nut (not shown) is fixed to the threaded second end 24 of the stem 16 after the plate (e.g., to the left of the plate in FIG. 6A). In such embodiments, the stem, by not being fixed to the plate, the non-articular lateral bony cortex 4, or the perimeter of the transhumeral tunnel 7, the stem is allowed movement along the axis of the stem within a range bounded by the nut. The nut limits the range of movement, in the second direction, away from the non-articular lateral bony cortex 4 and toward shell 15, but not towards the non-articular lateral bony cortex 4.

Alternatively, the stems 16 can capture either the non-articular lateral bony cortex 4 of the humerus or an optional washer-plate 30 and the shell 15 of the humeral implants 13, 17, serving to compress them together against the intervening elements of humeral bone 3, 4, 5 (FIGS. 10A, 10B). In some embodiments, screws 33 can be used through fastener cannulations 32 to help secure the washer-plate 30 to the non-articular lateral bony cortex 4 of the humerus (FIGS. 6A, 10A). The screws 33 can be used to compress the washer-plate 30 against lateral bony cortex 4 of the humerus 1 or can be fixed angle and locking to achieve rigid fixation to the humerus bone 1. Additional screws 33 can also bridge between fastener cannulations 32 in the washer-plate 30 and screw optional coupling sites 34 of the humeral implants 13, 17.

The portions of the shell 15 that interface with the bone may have a suitable protruding bony ingrowth surface(s) 21a to allow long-lasting adhesion to the humeral head 2 and metaphysis bone 5. Each stem 16 has two ends 23, 24 (FIGS. 5A, B). One end 23 removeably attaches to the coupling feature 22 of the shell 15 and another end 24 which engages the non-articular lateral bony cortex 4 of the humerus or the coupling site 31 of an optional washer-plate 30 fixed to the non-articular lateral bony cortex 4 of the humerus, or a nut, as explained above. In some embodiments, the remainder of the stem 16 may possess a bone ingrowth surface but ideally would not to facilitate slidable engagement and removal as necessary. Alternatively, the end 24 of the stem 16 may be smooth and allow slidable contact with the lateral washer-plate 30, the non-articular lateral bony cortex 4 and/or transhumeral tunnel 7 along a central axis in the metaphysis 5 of the humerus 1, as more fully explained above.

In some embodiments, the stem 16 has a cannulation 22n such as shown in FIG. 5L. The stem 16 has a first aperture 220 at a first end 23 in communication with the cannulation 22n and the second aperture 22p at a second end 24 in communication with the cannulation 22n. The cannulation 22n extends through the stem 16 from the first end 23 to a second end 24.

In some embodiments, the cannulation 22n is defined by one or more interior sidewalls that extend from the first end 23 to the second end 24. In some embodiments, the one or more interior walls are cylindrical throughout the cannulation and circular in cross-section. The cannulation 22n is co-axial with cannulations 22b, 22c, 22h, 22k of the articular humeral implants 13a, 17a and the spacer 120. The cannulation 22n may be at a radial center of the stem 16.

Each of the cannulations 22b, 22c, 22h, 22k, 22n, may be aligned and/or co-axial with one or more or all of the other cannulations 22b, 22c, 22h, 22k, 22n when used. Therefore, passage of items, such as of instruments, screws, and other implants or implant components, can be made continuously through the aligned and/or co-axial cannulations and the associated components comprising the cannulations.

In some embodiments, modular transhumeral reamers 80, 90 are utilized to prepare the humeral and glenoid surfaces 3, 42 for novel implant application (FIGS. 7A, 7B, 7C, 7D, 7E, 7F, 8A, 8B, 8C, 9A, 9B, 9C, 9D, 9E, 9F). Reamer 80, 90 comprises a cutting reamer head 81, 91 with an axle 55, a central cannulation 56, 93, and a handle 51. The handle 51 has a ring end 52 which captures the axle 55 and allows the axle 55 and the reamer head 81, 91 to spin freely in the ring end 52. The reamer head 81, 91 has a surface configured for bone drilling or cutting 83, 92, 96 and a slot 57, 98, opposite the cutting or drilling surface configured for temporary engagement with the tip 76 of the transhumeral reamer shaft 75. The shaft 75 of the reamers 80, 90 can be driven by hand or a power drill 77.

The reamers 80, 90 are modular and may be used through open surgical approaches used for traditional open shoulder arthroplasty surgery or they can be used for minimally invasive rotator cuff sparing shoulder arthroplasty surgery through a transhumeral tunnel 7 approach which spares the rotator cuff, and avoids dislocation of the shoulder joint associated with traditional approaches. For the transhumeral approach, the reamers 80, 90 are inserted by their handle 51 through a non-bony passageway, likely an interval in the rotator cuff, and into position in the shoulder joint while the transhumeral reamer shaft 75 is inserted through the transhumeral tunnel 7 and an optional protective transhumeral sheath 8 into the shoulder joint and reversibly coupled by the engagement tip 76 to the engagement slots 57, 98 of the reamer 80, 90.

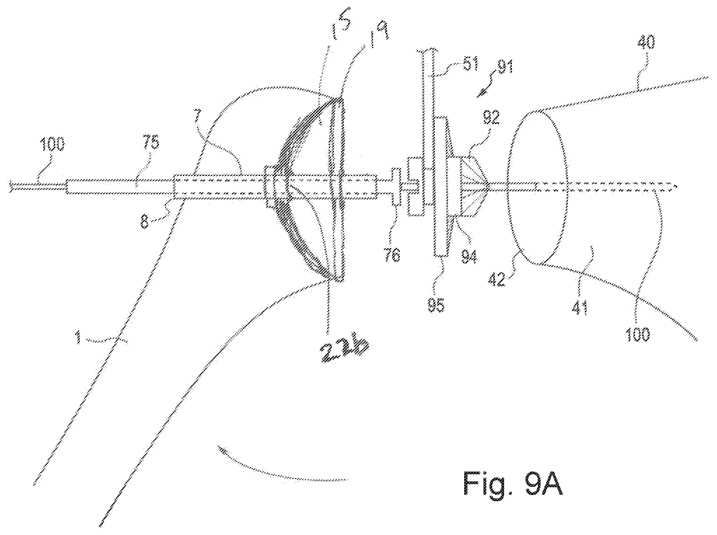

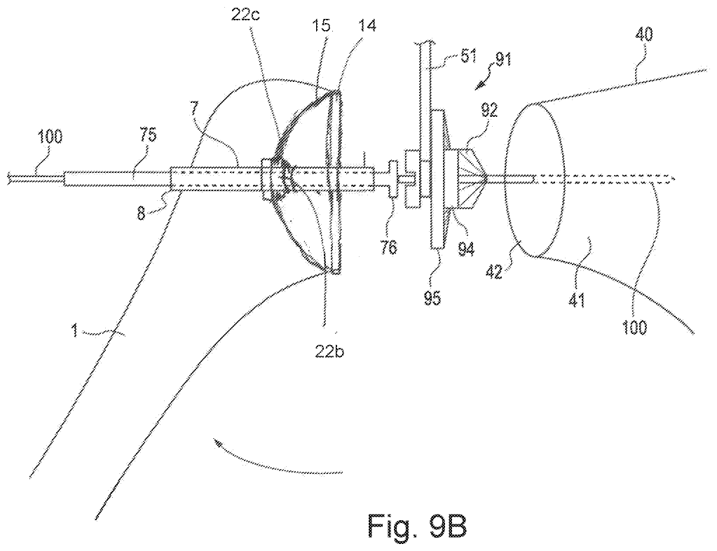

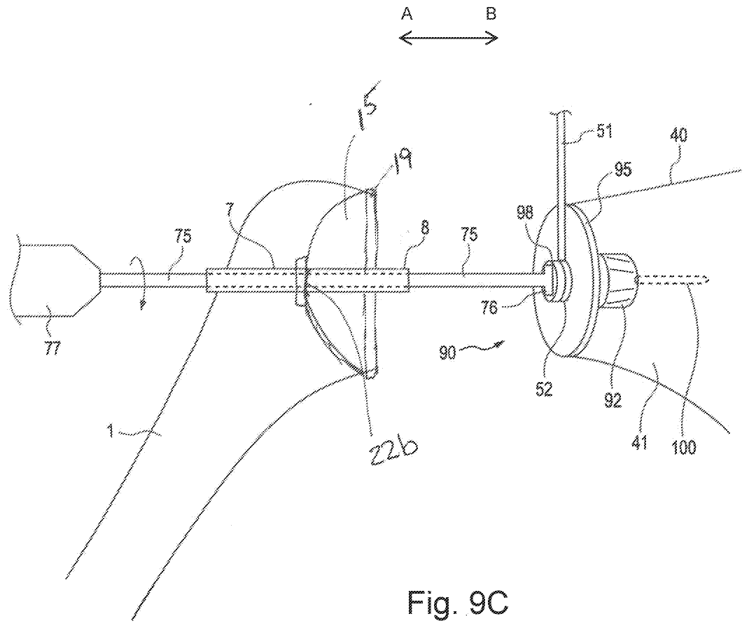

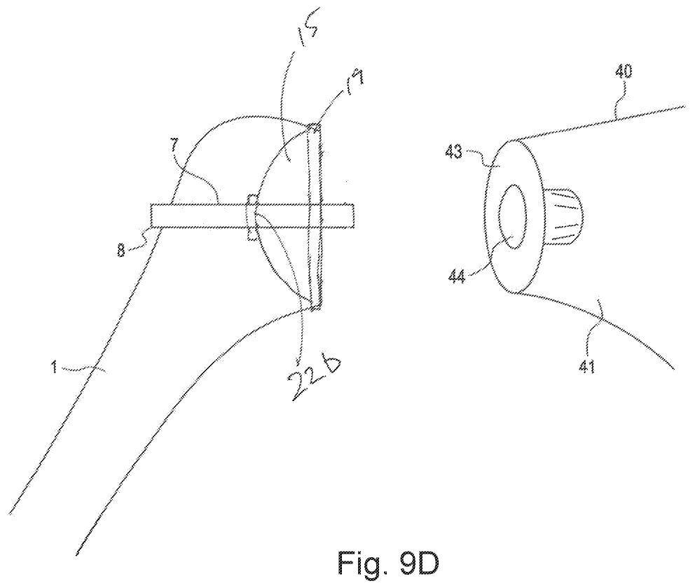

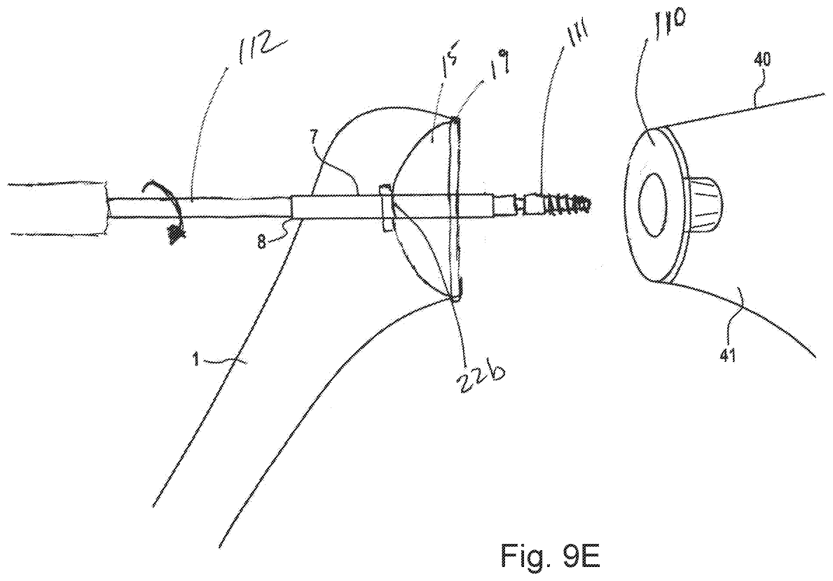

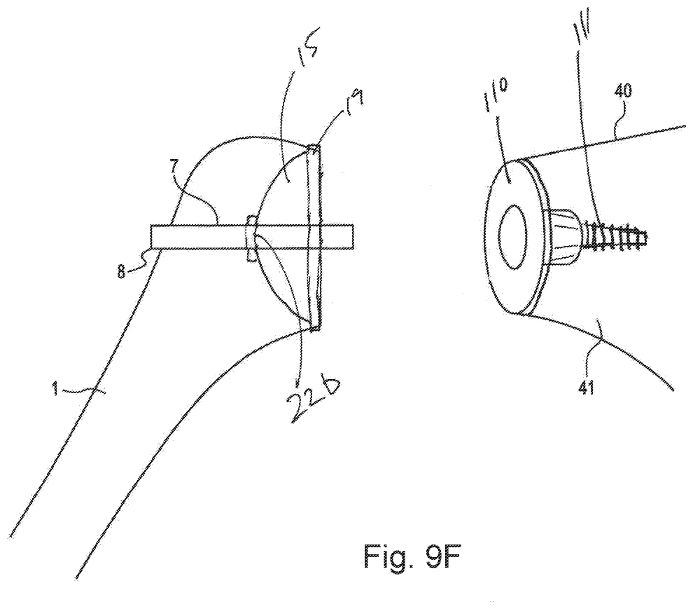

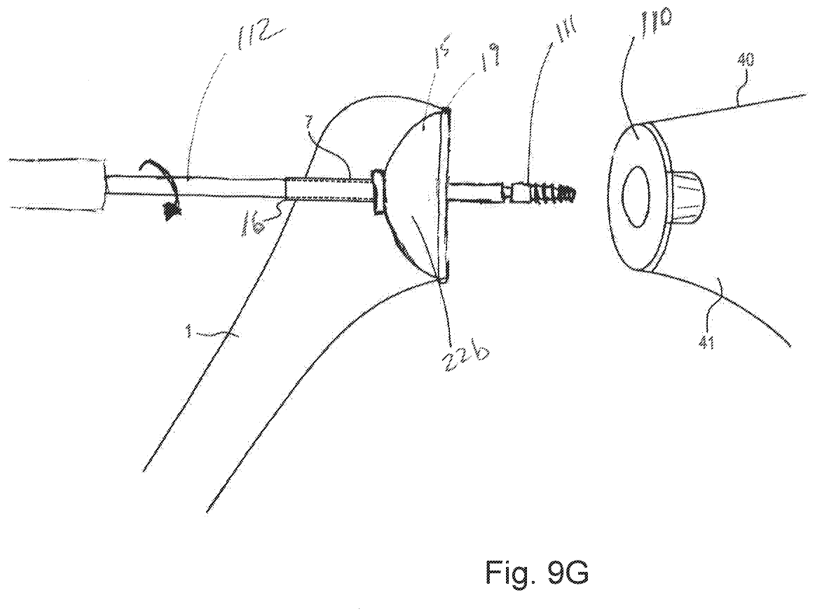

In some embodiments, when the glenoid reamer 90 is used, the shell 15 is placed or present in the prepared humeral head, then the transhumeral reamer shaft 75 is inserted through the transhumeral tunnel 7 and through the cannulation 22b of the shell 15 as shown in FIG. 9a, and optionally through cannulation 22c or 22h of the articular surface component 14, 18, if present. The optional protective transhumeral sheath 8 can be used at least between the lateral boney cortex 4 and the shell 15 or further into or through the cannulation(s) 22b, 22c, (and 22h, 22k, and/or 22n, when present) and beyond the shell 15, spacer 120, and/or articular surface component 14, 18 and into the shoulder joint, as shown in FIGS. 9B, 9C, and 9D. The shaft 75 can extend through the sheath as shown in FIG. 9A. While spacer 120 and articular surface component 18 are not shown in FIGS. 9A through 9G, the spacer 120 and or the articular surface component 18 could be provided within the shoulder joint and an instrument, such as the reamer shaft 75 could be further operated through their cannulations 22h, 22k. When a cannulated stem 16 is present, the instrument, such as the screw driver 112, could be further operated through its cannulation 22n, such as shown in FIG. 9G.

The reamer 80, 90 can be configured to be used to drill and cut transverse or perpendicular to the axis of the reamer shaft 75, particularly not off-axis. There is a centering sleeve 78 moving freely around the reamer shaft 75 that engages a centering cannulation 82 on the reamer head 81 to ensure the reamer cuts the humeral surface 3 on axis, i.e. perpendicular to the transhumeral tunnel 7 and the reamer shaft 75. The glenoid reamer 90 does not require a centering sleeve 78 but may be cannulated 93 to ream over an optional guide pin 100 positioned through the transhumeral tunnel 7, and optionally through the cannulation 22b of the shell when present, and optionally cannulations 22h, 22k, and/or 22n, when present, and the protective transhumeral sheath 8 and into the surface 42 and vault 41 of the glenoid 40. The reamer shaft for the glenoid may also possess a guide cannulation 74 for the guide pin 100.

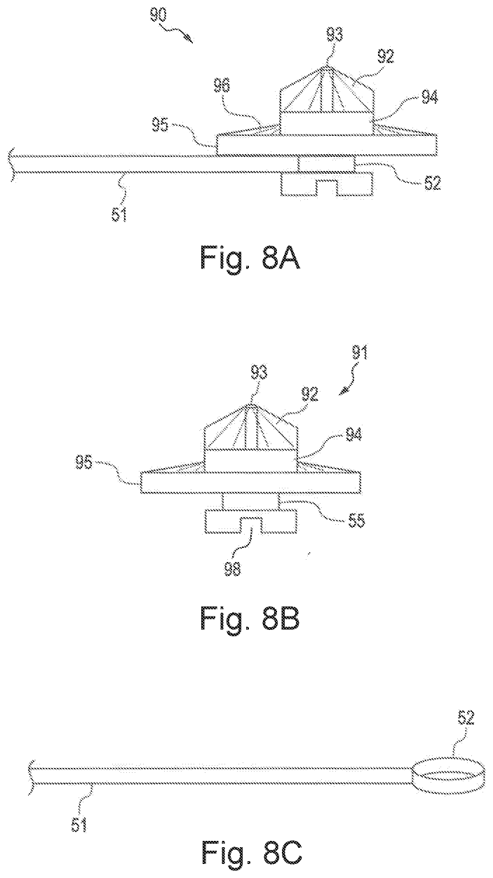

The cutting surfaces 83, 84, 92, and 96 vary in geometry and aggressiveness to optimally prepare the bone 3, 5, 41, 42 for their respective implants 13, 17, 110. The reamer head 81 for the convertible humeral implant 13,17 also has two cutting surfaces, one protruding cutting surface 83 with variable geometry to cut a humeral metaphyseal socket 85 and a second cutting surface 84 to cut the perimeter near the level 6 of the anatomic neck of the humerus 1 (FIGS. 7A, B, C, D, E, F). The cutting surface 92, 96 of the glenoid reamer 90 has a protruding aggressive cutting surface 92 to cut a precise glenoid cavity 44 into the glenoid vault 41; an intermediate smooth non-cutting surface 94 to help keep the reamer 90 on axis and prevent damage to prepared peripheral glenoid surface bone 43; and a less aggressive cutting surface 96 on the peripheral rim 95 of the glenoid reamer head 91 to less aggressively prepare the peripheral aspect of the glenoid surface 42 (FIGS. 8A, B, C; 9 A, B, C, D, E, F).

In some embodiments, use of transhumeral instrument operation through the tunnel 7 and the cannulation(s) 22b, 22c, 22h, 22k, and/or 22n may provide minimally invasive techniques and limited exposures to the joint. For example, when performing a shoulder replacement without dislocating the shoulder or cutting the rotator cuff, the space available for inserting humeral and glenoid implants is limited. And therefore, it may be advantageous to place the bone fixation component 15 and/or entire articular humeral component 13a, 17a with or without a spacer 120 and/or stem 16 before the steps of preparing the glenoid or inserting and fixing some of the glenoid components.

In some embodiments, transhumeral instruments can traverse the transhumeral tunnel 7 and the cannulation(s) 22b, 22c, 22h, 22k, 22n in the bone fixation component 15 and/or articular surface component 14, 18 and/or the spacer 120 and/or the stem 16, when present, to prepare the glenoid or insert or repair/replace the glenoid implant or a portion thereof.

In some embodiments, the implants 13 and/or 17, and/or 110 can be implanted through an implant method or shoulder replacement method that comprises the following steps: placing a component of a humeral implant 13, 17 comprising a cannulation, at prepared portion 9, 85, 7 of a proximal humerus 2a; and, operating an instrument transhumerally through the cannulation. In some embodiments, the component is the stem 16 with cannulation 22n, the bone fixation component 15 with cannulation 22b, the articular surface component 14, 18 with or without cannulation, and/or the spacer 120. In some embodiments, the instrument is operated at the glenoid transhumerally through the cannulation(s).

In some embodiments, the step of placing comprises placing a bone fixation component 15 of a humeral implant 13, 17, comprising a cannulation 22b at prepared portion 9, 85 of a proximal humerus 2a; and, the step of operating comprises operating an instrument transhumerally through the cannulation 22b.

In some embodiments, the step of placing a component of a humeral implant 13, 17 comprising a cannulation, at prepared portion of a proximal humerus 2a comprises the step of fixing the bone fixation component 15 to the prepared portion of the proximal humerus.

In some embodiments, a transhumeral tunnel 7 (FIG. 9D) is cut by a drill driving a drill bit through the proximal humerus before, after, or at the time the bone fixation component 15 is placed at the proximal humerus. The cannulation 22b is aligned with the transhumeral tunnel 7. When the cannulation 22c, 22h is present in the articular surface component 14, 18 and/or the cannulation 22k in the spacer 120 and/or the cannulation 22n in the stem 16 the cannulations 22c or 22h and 22k, 22n are aligned with the transhumeral tunnel 7 and the cannulation 22b.

In some embodiments, the step of operating comprises preparing a glenoid 40 to receive a glenoid implant 110. In some embodiments, the step of preparing comprises cutting a glenoid cavity 44 into the glenoid vault 41 of the glenoid 40, such as shown at FIGS. 9A, 9B, 9C, and 9D. In some embodiments, the instrument is the reamer 90 and it is operated to cut the glenoid cavity 44 into the glenoid vault 41 to receive the glenoid implant. In some embodiments, the instrument comprises a shaft 75 and the glenoid reamer 90. The shaft extends into and through the humerus, the tunnel 7, and the cannulation 22b, optionally the cannulation 22c, 22h when the articular surface component 14, 18 is present, optionally the cannulation 22k when the spacer 120 is present, and optionally the 22n cannulation when the stem 16 is present, to the glenoid reamer. In some embodiments, the step of cutting comprises driving the shaft 75 to rotate with a power drill 77.

In some embodiments, the step of preparing comprises the following. A reamer head 91 of the reamer 90 is placed in a shoulder joint through a non-bony passageway. A shaft 75 is placed into and through the humerus, the tunnel 7, and the cannulation 22b, optionally the cannulation 22c, 22h when the articular surface component 14, 18 is present, optionally the cannulation 22k when the spacer 120 is present, and optionally the 22n cannulation when the stem 16 is present. In some embodiments, the step of removing a first glenoid implant comprises drilling into or through the first glenoid implant with a drill. Therefore, the cannulation 22b (and cannulations 22c, 22h when the articular surface component 14, 18 is present, optionally the cannulation 22k when the spacer 120 is present, and optionally the 22n cannulation when the stem 16 is present) can be used to implant, revise, remove, or exchange components of a glenoid implant by operating an instrument transhumerally through the tunnel 7 and cannulations 22b, 22c, 22h, 22k, 22n and/or by-passing implant components through the tunnel 7 and cannulations 22b, 22c, 22h, 22k, 22n.

In some embodiments, the step of operating comprises placing or fixing a glenoid implant 110 to a prepared glenoid. The step of placing or fixing may comprise placing or fixing a component of the glenoid implant, such as a fastener, such as a screw 111, into the glenoid implant, by placing the component through the tunnel 7 and the cannulation(s) 22b, (and cannulations 22c, 22h when the articular surface component 14, 18 is present, the cannulation 22k when the spacer 120 is present, and the 22n cannulation when the stem 16 is present) with an instrument such as a screw driver 112 and rotating the screw into the glenoid implant 110 with a screw driver 112. The screw driver 112 may be hand turned or may be driven by a drill or motor. In some embodiments, the step of fixing or placing comprises rotating a screw 111 until the screw 111 is seated in the glenoid implant and contacts glenoid bone, such as shown in FIGS. 9E, 9F, and 9G.

After use of the instrument, such as the reamer, drill, screw driver, threaded tipped shaft, or other instrument is complete, the instrument is removed from the cannulation(s) 22b and optionally the cannulations 22c, 22h, 22k, 22n when present by withdrawing the instrument in the direction A (FIG. 9C).

In some embodiments, the articular surface component 14, 18 comprises a second cannulation 22c, 22h, and in such case, the articular surface component 14, 18 can be fixed to the bone fixation component 15 before or after the instrument is operated at a glenoid transhumerally through the cannulation 22b. Then the instrument can be operated at a glenoid transhumerally through the first cannulation 22b and the second cannulation 22c, 22h. Further, the instrument can be operated transhumerally through the first cannulation 22b to fix or assist in fixing the articular surface component 14, 18 to the bone fixation component 15, with or without the use or presence of a second cannulation 22c, 22h in the articular surface component 14, 18.

In some embodiments, the instrument is operated transhumerally through the cannulation 22b and then the instrument is removed from the cannulation 22b and the tunnel 7, and then the articular surface component 14, 18 is fixed to the bone fixation component 15.

In some embodiments, the articular surface component 14, 18 is fixed to, placed at or adjacent the bone fixation component and the instrument is operated transhumerally through the cannulations 22b, 22c, 22h.

In some embodiments, fixing the articular surface component of the humeral implant to the bone fixation component or the spacer 120, if used, comprises operating the instrument in and/or through the cannulation 22b in the bone fixation component (and cannulation 22k if a spacer 120 is used), to fix the articular surface component and/or spacer. This can include attaching an instrument, such as by threading (either directly or with a screw) to the back of the articular surface component or spacer, and back-slap driving (hitting the instrument in the direction opposite of the glenoid), to press fit the articular surface component and/or spacer into the bone fixation component (or spacer, if used). If a screw is used to connect to the back of the articular surface it could be left in the articular surface component or removed via the cannulation(s) after the articular surface component is seated on the bone fixation component or spacer. In some embodiments, the cannulation 22b may not be used to fix the articular surface component of the humeral implant to the bone fixation component or the spacer, and the instrument is withdrawn from the cannulation 22b before the articular surface component is fixed.

In some embodiments, the spacer 120 is fixed to, placed at or adjacent the bone fixation component and the instrument is operated transhumerally through the cannulations 22b, 22k.

In some embodiments, the spacer 120 is fixed to, placed at or adjacent the bone fixation component and the articular surface component 14, 18 is fixed to, placed at or adjacent the spacer 120, and the instrument is operated transhumerally through the cannulations 22b, 22c, 22h, 22k. In some embodiments, the stem is fixed to, placed at or adjacent the bone fixation component, opposite of the articular side of the bone fixation component, and the instrument is operated transhumerally through the cannulations 22n, 22b, 22c, 22h, 22k.

In some embodiments, the stem is fixed to, placed at or adjacent the bone fixation component, opposite of the articular side of the bone fixation component, and the instrument is operated transhumerally through the cannulations 22n, 22b and optionally through cannulation 22c, 22h, 22k.

In some embodiments, the step of placing comprises placing the stem 16 in the tunnel 7 within the head 2 of the proximal humerus 2a; and, the step of operating comprises operating an instrument transhumerally through the cannulation 22n. The instrument may be operated through the stem cannulation 22n only or through the stem cannulation and the cannulation(s) 22b, 22c, 22h, and/or 22k of one or more other components, such as the bone fixation component 15 and/or the articular surface component 14, 18 and/or the spacer 120. The operating may be at the glenoid and or as otherwise described above. For example, the operating may be to remove, connect or fix another component of the humeral implant or a glenoid implant or to prepare the corresponding bone(s) for the same.

As has been shown herein, work, operations, and manipulation can be performed in and through the cannulation(s).

Further, in some embodiments, when a stem 16 is used, the stem is connected to the bone fixation component 15. The connecting may occur by placing the stem 16 in the tunnel 7 in the proximal humerus. Further, in some embodiments, the connecting step is not used, for example, when the stem is already fixed to the bone fixation component or when a stem is not used.

However, the reversibly connected stem 16, cannulated or not, also protects the transhumeral tunnel and keeps it open and clear from postoperative changes and obstruction, maintaining it for later potential revision needs and transhumeral instruments for humeral and glenoid implant removal.

The articular surface component 14, 18 may be fixed to the bone fixation component 15 at or after the articular humeral component 13a, 17a is seated on the prepared articular portion of the humerus.

In some embodiments, the glenoid implant 110 is placed at the glenoid, such as at the glenoid vault, before the step of placing the bone fixation component 15, of a humeral implant 13, 17, at the prepared portion 9, 85 of a proximal humerus bone 1. Then the bone fixation component 15 of a humeral implant 13, 17, is placed at the prepared portion 9, 85 of a proximal humerus bone 1, Then the instrument is operated at a glenoid transhumerally through the cannulation 22b and/or 22c, 22h, 22k or 22n, when present, as explained above.

Depending on the chosen approach for implantation, the order of the foregoing steps can vary.

In some embodiments, the stem is placed through tunnel 7 from the lateral bony cortex 4 side in the direction C and is connected to the bone fixation component of the articular humeral component 13a, 17a before the bone fixation component 15, with or without the articular surface component 14 and 18 or spacer 120, when used, coupled, is fixed to the prepared articular portion 9, 85 of a proximal humerus 1. In some embodiments, the stem 16 is placed in the tunnel 7, the bone fixation component 15 with or without the articular surface component 14 and 18 coupled, is fixed to the prepared articular portion 9, 85 of a proximal humerus 1, and then the stem 16 is connected to the bone fixation component 15.

In some embodiments, the step of connecting the stem occurs during the step of placing. For example, the stem 16 may be connected to the bone fixation component 15 when it is placed within the tunnel 7. As explained above, in some embodiments, the stem 16 is placed in the tunnel 7 in the proximal humerus 1 without fixing the stem 16 within the tunnel 7 against post-surgery longitudinal movement.

Some of the non-limiting benefits and/or features of at least some embodiments of the humeral implant 13, 17 and method disclosed herein comprise the following: providing a novel humeral implant for shoulder replacement surgery that can be implemented with minimally invasive novel surgical techniques; providing a humeral implant which includes an optional stem that resides along a central axis of the proximal humerus rather than in the intramedullary canal; providing a humeral implant which preserves humeral bone stock and allows increased physiologic load transmission from the joint surface in a patient's shoulder along the bone of the proximal humerus; providing a humeral implant which has an interchangeable articular surface components and allows conversion between an anatomic shoulder arthroplasty and a reverse shoulder arthroplasty; providing a modular humeral implant which can be used to help prepare a glenoid process in a shoulder joint without transecting their rotator cuff tendon and/or without dislocating their shoulder joint; providing a modular humeral implant which can be used to prepare a glenoid process in a shoulder joint using a reamer and other instruments through a transhumeral passage in line with the cannulation(s) of the humeral implant; and/or providing a modular humeral implant which can be used to revise or convert humeral and glenoid implants utilizing instruments through a transhumeral passage in line with the cannulation of the humeral implant.

It is therefore intended that the foregoing detailed description be regarded as illustrative rather than limiting, and that it be understood that it is the following claims, including all equivalents, that are intended to define the spirit and scope of this invention.

1. A humeral implant comprising:
a bone fixation component configured for fixation to an articular portion of a proximal humerus and comprising a cannulation extending through the bone fixation component; and,
an articular surface component connectable to the bone fixation component and comprising an articular surface.

2. The humeral implant of claim 1, wherein the bone fixation component comprises a bone fixation side and an articular surface component side, the cannulation extends through the bone fixation component from the bone fixation side to the articular surface component side.

3. The humeral implant of claim 1, wherein the cannulation comprises a circular cross-section.

4. The humeral implant of claim 1, comprising a stem; and, the cannulation is co-axial with the stem.

5. The humeral implant of claim 4, wherein an interior wall of the cannulation at a terminal end at a bone fixation side of the bone fixation component comprises a first threads, and an end of the stem comprises a second threads, the stem is connectable at the interior wall between the first threads and the second threads.

6. The humeral implant of claim 4, wherein the stem is sized to frictionally fix within the cannulation.

7. The humeral implant of claim 4, wherein the stem comprises a female configuration or a male configuration to connect with the other of a male configuration or a female configuration of the bone fixation component.

8. The humeral implant of claim 4, wherein the cannulation is a first cannulation, and the stem comprises a second cannulation and the second cannulation is co-axially aligned with the first cannulation when the stem is connected to the bone fixation component.

9. The humeral implant of claim 4, wherein the stem is configured to extend along a central axis of a neck of a humerus.

10. The humeral implant of claim 4, wherein the stem comprises a length sufficient to traverse a non-articular lateral bony cortex of a humerus opposite the articular surface component.

11. The humeral implant of claim 1, wherein the cannulation is a first cannulation; and the articular surface component comprises a second cannulation, the second cannulation is aligned with the first cannulation when the articular surface component is connected to the bone fixation component.

12. The humeral implant of claim 11, wherein the articular surface component comprises an articular side and a connecting side, the second cannulation extends through the articular surface component from the connecting side to the articular side.

13. The humeral implant of claim 1, wherein the cannulation comprises a width of 1 millimeter to 25 millimeters.

14. The humeral implant of claim 1, wherein the cannulation is located at a center of the bone fixation component.

15. The humeral implant of claim 1, wherein the cannulation is a first cannulation and the bone fixation component comprises a fastener cannulation.

16. The humeral implant of claim 1, wherein the cannulation is configured to be aligned with a transhumeral tunnel in the proximal humerus.

17. The humeral implant of claim 1, comprising a stem, and a washer plate for engaging a lateral non-articular bone surface of a humerus, the cannulation is co-axial with the stem, the washer plate is configured to receive the stem, the stem is axially slidably received by the washer plate.

18. The method of shoulder replacement, comprising the step of:
placing a humeral implant component comprising a cannulation at a prepared portion of a proximal humerus; and,
operating an instrument transhumerally through the cannulation.

19. (canceled)

20. The method of claim 18, wherein the step of operating comprises preparing a glenoid to receive a glenoid implant.

21.-41. (canceled)

42. A humeral implant comprising:
an articular humeral component configured for fixation to an articular portion of a proximal humerus and comprising a bone fixation side, an articular surface, and a cannulation extending through the articular humeral component from the bone fixation side through the articular surface.

43. The humeral implant of claim 42, wherein the cannulation comprises a first cannulation and a second cannulation; the articular humeral component comprises a bone fixation component and an articular surface component; the bone fixation component comprises the first cannulation, the articular surface component comprises the second cannulation and the articular surface, the second cannulation intersects the articular surface, the first cannulation and the second cannulation are aligned co-axially.

44. A humeral implant stem comprising:
an elongated body comprising a first end, a second end, a coupling portion, and a cannulation, the cannulation extending through the elongated body and open to the first end and the second end, the coupling portion is at the first end or the second end.
